(19) United States
(12) Patent Application Publication
(10) Pub. No.: US 2002/0025306 A1
(43) Pub. Date: Feb. 28, 2002

(54) Methods of increasing the glucose responsiveness of pancreatic ss-cells

(76) Inventors: Edward E. Baetge, St. Sulpice (CH); William Pralong, Lausanne (CH); Hamid Hussain Khan, Sullens (CH)

Correspondence Address: MINTZ, LEVIN, COHN, FERRIS,, GLOVSKY and POPEO, P.C., One Financial Center, Boston, MA, 02111, US

(21) Appl. No.: 09/755,539

(22) Filed: Jan. 5, 2001

Related U.S. Application Data
(60) Provisional application No. 60/175,021, filed on Jan. 7, 2000.

(51) Int. Cl. A61K 48/00; C12N 5/08

(52) U.S. Cl. 424/93.21; 435/366

(57) ABSTRACT

The instant invention involves methods of increasing the glucose responsiveness of pancreatic β-cells by providing a source of a glucoincretin. Also provided are methods of treating a disorder characterized by impaired β-cell function by providing a population of β-cells and a source of a glucoincretin. Additionally, the invention pertains to a population of purified β-cells and a kit for use in the treatment of a disorder characterized by impaired function.

### RELATED APPLICATIONS

This application claims priority to U.S. Ser. No. 60/175,021, filed Jan. 7, 2000, which is incorporated herein by reference.

### FIELD OF THE INVENTION

This invention relates generally to the fields of molecular biology and endocrinology.

### BACKGROUND OF THE INVENTION

Diabetes mellitus is a chronic disorder of carbohydrate metabolism characterized by insufficient production of insulin by the β-cells of the pancreas. Diabetes affects approximately 10 million people in the United States, and more than 250,000 new cases are diagnosed each year. The two common types of diabetes mellitus are Type I diabetes (also known as insulin-dependent diabetes) and Type-II diabetes (also known as non-insulin-dependent diabetes). Insulin-dependent diabetes is generally characterized by an absolute deficiency of insulin production, whereas non-insulin-dependent diabetes is characterized by a relatively insufficient production of insulin.

In normal individuals, the rate of insulin secretion by beta cells is regulated by the level of glucose in the blood. When blood glucose levels rise, the β-cells are stimulated to release increased amounts of insulin into the blood, thereby accelerating glucose transport into the cells and the conversion of glucose into glycogen. As blood glucose levels fall, insulin release from the β-cells is decreased. In diabetic subjects, insulin production is abnormally low or insufficient, which results in abnormally high blood glucose levels (i.e. hyperglycemia).

Current insulin-dependent diabetes treatment methods include diet and exercise programs as well as the constant and life-long monitoring of blood glucose levels in conjunction with injections of insulin. However, many diabetic subjects experience difficulty controlling their blood glucose levels using the currently available treatment methods. Thus, they are constantly exposed to the adverse effects of hypoglycemia (abnormally low blood glucose levels) as well as hyperglycemia.

The inability to precisely control blood glucose levels also poses long term complications including degenerative vascular changes (e.g. atherosclerosis and microangiopathy), neuropathy (e.g. peripheral nerve degeneration, autonomic nervous system, and cranial nerve lesions), ocular disturbances (e.g. blurred vision, cataracts, and diabetic retinopathy), kidney diseases (e.g. recurrent pyelonephritis and nephropathy), and infections. Accordingly, there exists a need for an alternative method for controlling blood glucose levels in the diabetic patient. The transplantation of beta cells has been proposed as an alternative therapy in the treatment of diabetes.

### SUMMARY OF THE INVENTION

In one aspect, the invention concerns a method of enhancing the responsiveness of β-cells to glucose by providing a source of a glucoincretin that increases the responsiveness. In one embodiment, the source of the glucoincretin is an endogenous glucoincretin gene. In a further embodiment, the β-cells are transfected with this endogenous glucoincretin gene. In an alternative embodiment, the glucoincretin source is exogenous to the cell. The exogenous source may be delivered into the β-cells by a transport peptide. In another embodiment, the β-cells are isolated.

In a still further embodiment, the glucoincretin is a cAMP-raising agent. Examples of suitable cAMP-raising agents include, but are not limited to IBMX, GLP-1, GIP, glucagon, a cAMP-raising drug, a cAMP-raising enzyme, and a cAMP-raising hormone.

In another aspect, the invention is concerned with a method of treating a disorder characterized by impaired β-cell function by supplying a population of β-cells and a source of a glucoincretin to a patient suffering from the disorder. In one embodiment, the β-cells and the source of the glucoincretin are encapsulated within a bioartificial organ. This bioartificial organ may contain a semipermeable jacket and a core containing both the β-cells and the source of the glucoincretin.

The source of the glucoincretin can alternatively be either an endogenous glucoincretin gene or it can be an exogenous source. When endogenous, one, some, or all of the β-cells are transfected with the endogenous gene. When exogenous, the source can be delivered into the β-cells by a transport peptide. The glucoincretin can, in one embodiment, be a cAMP-raising agent such as IBMX, GLP-1, GIP, glucagon, a cAMP-raising drug, a cAMP-raising enzyme, or a cAMP-raising hormone.

In a further aspect, the invention concerns a population of purified β-cells transfected with an endogenous glucoincretin gene.

In a still further aspect, the invention describes a kit made up of a population of purified β-cells and a source of a glucoincretin for use in the treatment of a disorder characterized by impaired β-cell function. In various embodiments, the source may be endogenous or exogenous and the glucoincretin may be a cAMP-raising agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a diagram showing the effect of GLP-1 injection in non-fasted diabetic mice implanted with encapsulated CDM3D cells in flatsheets or agarose discs.

FIG. 2 is a diagram showing the effect of GLP-1 secreting β-cells on the membrane potential of β-tcTet cells.

FIG. 3 is a diagram showing the effect of GLP1-gly8 and Exendine-(9-39) on the membrane potential of β-tcTet cells.

FIG. 4 is a diagram showing the effect of Exendine-(9-39) and GLP-1-gly8 on the membrane potential of β-tcTet cells.

FIG. 5 is a diagram showing the effect of GLP-1-secreting β-cells and Exendine-(9-39) on membrane potential of β-tcTet cells.

FIG. 6 is a diagram showing the effect of Exendine-(9-39) and the supernatant of GLP-1 secreting β-cells on membrane potential of β-tcTet cells.

FIG. 7 is a diagram showing the effect of Exendine-(9-39) and the supernatant of GLP-1 secreting β-cells on membrane potential of β-tcTet cells.

### DETAILED DESCRIPTION OF THE INVENTION

Diabetes is characterized by an insufficiency of the pancreatic β-cells to maintain normoglycemia. Under normal conditions, the number of β-cells is positively correlated with body mass. In diabetic patients, the number of β-cells is reduced. Thus, in order to effectively treat diabetes, it is important to improve the function of the β-cells as well as the number of β-cells in a patient.

The endocrine secretions of the pancreatic islets (i.e, β-cells) are under complex control not only by blood-borne metabolites such as glucose, amino acids, catecholamines, etc., but also by local paracrine influences. The major pancreatic islet hormones (i.e, glucagon, insulin, and somatostatin) interact among their specific cell types in order to modulate the secretory responses mediated by the metabolites. Although insulin secretion is predominantly controlled by blood glucose levels, glucagon and somatostatin stimulate and inhibit glucose-mediated insulin secretory responses, respectively. See U.S. Pat. Nos. 5,118,666; 5,120,712; 5,545,618; and 5,614,492, each of which is incorporated herein by reference.

Glucoincretins, including glucagon and GLP-1 (glucagon like peptide-1), have been shown to stimulate glucose-induced insulin release and insulin biosynthesis. They also act to restore the glucose competence of the β-cells as well as to increase the number and/or size of β-cells. See WO 00/07617, incorporated herein by reference. GLP-1 has been shown to have insulinotropic properties and may delay gastric emptying. By “insulinotropic properties” is meant that GLP-1 stimulates insulin secretion, i.e, it is a glucoincretin. In addition, GLP-1 stimulates cAMP formation, which, in turn, leads to increased insulin release. See Mojsov, Int. J. Peptide Protein Research, 40:333-43 (1992).

As used herein, the terms “glucoincretin”, “insulinotropic properties”, and “insulinotropic activity” are intended to refer to the ability of an agent to increase β-cell responsiveness to increased blood glucose levels.

In the field of β-cell physiology, glucoincretin stimulation is one key component for the proper functioning of β-cells. GLP-1, is a peptide hormone that controls the glucose responsiveness of β-cells. In the short term, this is accomplished by modulating the glucose sensing ability of the β-cells and by modulating exocytosis. In the long term, GLP-1 modulates β-cell pro-insulin gene expression. In vivo, this modulation is achieved by the release of GLP-1 into the blood stream from L-cells of the intestinal epithelium in response to stimulation by entry of the gastric contents. GLP-1, in turn, will reach the pancreas, where it binds to the GLP-1 receptors on the surface of the β-cells.

Post-prandially (e.g., after ingestion of a meal), GLP-1 receptor activation stimulates increased intracellular production of cAMP. This, in turn, leads to increased insulin secretion, which is the result of both enhanced closing of ATP sensitive potassium (K⁺ ATP) channels in the membrane of the β-cell as well as protein kinase A activation of distal exocytotic events.

The human hormone glucagon, another glucoincretin, is a 29-amino acid hormone produced in the pancreatic A-cells. When glucagon binds to its receptor on insulin producing β-cells, cAMP production increases. See U.S. Pat. No. 5,512,549, incorporated herein by reference. This, in turn, stimulates insulin expression. See Korman et al., Diabetes 34:717-22 (1985). High levels of insulin down-regulate glucagon synthesis by a feedback inhibition mechanism. See Ganong et al., Review of Medical Physiology, Lange Publications, Los Altos, Calif., p. 273 (1979). Therefore, the expression of glucagon is carefully regulated by insulin, and, ultimately, by serum glucose levels.

When a purified primary β-cell, or a differentiated β-cell line is stimulated in vitro with elevated glucose concentrations, the β-cells will not respond at all (or only weekly respond with respect to insulin secretion) in the absence of costimulation by a glucoincretin. See Holz et al., Nature 361(6410):362-365 (1993); Fehmann et al., Endocrine Reviews 16:390-410 (1995); and Thorens et al., Diabetes 42:1219-25 (1993), each of which are incorporated by reference.

The correction of diabetes secondary to direct engraftment under the kidney capsule of pure β-cells or intra peritoneal (IP) implantation following microencapulation in PLL-alginate indicates that these cells can obtain sufficient levels of incretins, thereby insuring proper insulin secretion under these conditions.

In contrast, if purified (fully differentiated) β-cells are macroencapsulated into bioartificial organs having thermoplastic biocompatible membranes and subsequently transplanted in vivo, the intimate contact with the blood supply is insufficient to maintain the gluco-competency of the β-cells. By “gluco-competency” is meant the ability of the β-cells to respond appropriately to an increase in blood glucose levels. Thus, for macroencapsulation, the addition of a glucoincretin priming signal is required to enable the encapsulated β-cell preparation to be gluco-competent (both in vivo and in vitro). In the absence of glucoincretin co-stimulation, the implanted β-cells will not be gluco-competent and will not survive.

Examples of suitable glucoincretins according to this invention include, but are not limited to, cAMP-raising agents such as IBMX, GLP-1, GIP, glucagon, a cAMP-raising drug, a cAMP-raising enzyme, and a cAMP-raising hormone. One skilled in the relevant arts will recognize that any cAMP-raising agent can be used to increase the glucose responsiveness of β-cells. In one preferred embodiment, the glucoincretin is GLP-1.

Moreover, the inclusion of an endogenous glucoincretin gene that is expressed within the purified β-cells can markedly increase the survival properties of the macroencapsulated cells upon transplantation in vivo. Specifically, non-GLP-1 expressing β-cells do not survive after implantation, whereas GLP-1 expressing β-cells do survive in vivo. The transplantation of a mixed population of encapsulated cells (both GLP-1 expressing and non-GLP-1 expressing β-cells) results in the overall cell survival of both cell types after transplantation in vivo.

Method of Enhancing Responsiveness of β-cells to Glucose

In one aspect, the invention provides a method of enhancing the responsiveness of β-cells to glucose by providing a source of a glucoincretin to the β-cells. The glucoincretin acts to increase the responsiveness of the β-cells to glucose. For example, the glucoincretin may be a cAMP-raising agent. Such agents include, but are not limited to IBMX, GLP-1, GIP, glucagon, a cAMP-raising drug, a cAMP-raising enzyme, and a cAMP-raising hormone. Other suitable cAMP-raising agents (or other glucoincretins) may also be supplied. Determination of suitable glucoincretins (i.e, insulinotropic agents) may be made by people of ordinary skill in the relevant art according to the methods described in Example 1, infra. Moreover, other methods known to those skilled in the art may also be used.

As used herein, the term “enhancing the responsiveness of β-cells to glucose” refers to the ability of the β-cells to appropriately respond to increased levels of glucose in the blood stream. Specifically, as blood glucose levels increase, a β-cell with increased responsiveness to glucose will lead to increased insulin secretion from the β-cells.

The source of the glucoincretin may be either endogenous or exogenous to the β-cells. When the source of the glucoincretin is endogenous, the β-cells may be transfected with an endogenous glucoincretin gene. Any method of transfection known to those skilled in the art can be used to accomplish this.

A number of well-known methods exist for introducing genetic material into target cells. These include the use of polycations such as DEAE-dextran (see McCutchan, et al., J. Natl. Cancer Inst. 41:351-57 (1968) and Kawai et al., Mol. Cell. Biol. 4:1172-74 (1984)); calcium phosphate coprecipitation (see Graham et al., Virology 52:456-67 (1973)); electroporation (see Neumann et al, EMBO J. 7:841-45 (1982)); lipofection (see Felgner et al., Proc. Natl. Acad. Sci. USA 84:7413-17 (1987)); retrovirus vectors (see Cepko et al., Cell 37:1053-62 (1984)); and microinjection (see Capecchi et al., Cell 22:479-88 (1980)).

When the source of the glucoincretin is exogenous to the β-cells, the glucoincretin may be delivered into the β-cells by means of a transport peptide. Those skilled in the art will recognize that any number of suitable transport peptides may be used to deliver the exogenous source of the glucoincretin into the β-cells.

For example, VP22 is a structural protein found in Herpes simplex type 1 virus (HSV). The herpesviral HSV-1 virion protein VP22 possesses an unusual intercellular trafficking mechanism (see WO 97/05265; Elliott & O'Hare, 88 Cell 223-233 (1997)). The protein can efficiently transport itself through the membrane of cells via a non-classical Golgi-independent mechanism. The VP22 protein can transport itself into surrounding cells as the result of endogenous synthesis and secretion or after exogenous application to naive cells. VP22 can spread throughout a monolayer of non-expressing cells, whereby VP22 is transported from the cytoplasm of an expressing cell into neighboring cells. Interestingly, the VP22 protein is naturally targeted to the nucleus where it binds directly to chromatin and segregates to daughter cells after cell division. Furthermore, when fused to a variety of other proteins the VP22 protein can transport the fused proteins across cell membranes thus carrying the attached proteins into the nucleus. More importantly, the VP22-fused proteins have been shown to retain biological activity in their chimeric state and to deliver this activity directly into the exposed cell in a highly efficient manner.

This VP22-fusion protein transport capability has recently been demonstrated for a variety of different proteins including Green Fluorescent protein, a 27 Kda fluorescent marker protein, (see Elliott & O'Hare, 6 Gene Therapy 149-151, 1999); P-53, a 53 Kda cell cycle regulatory protein, (see Phelean et al., 16 Nature Biotechnology 440-443, 1998); Thymidine Kinase, the 52 Kda enzyme serving as the converting enzyme in the pro-drug suicide protein combination routinely used in gene therapy trials; (see Dilber et al., 6 Gene Therapy 12-21 (1999)), and β-galactosidase, the 116 Kda bacterial enzyme widely employed as a reporter protein in gene expression studies (Invitrogen). In all of these studies the chimeric VP22 fusion proteins were efficiently transported into fusion-protein exposed cells, and most importantly, demonstrated the biological effects associated with each coupled protein both in vitro and also in vivo for the VP220TK system.

Various other proteins have the capability to permeate cellular membranes by the addition of a membrane-translocating sequence (MTS). See Rojas et al., 16 Nature Biotechnology 370-375 (1998). The MTS, a hydrophobic region (h-region) is used to deliver various peptides and proteins (cargo) across cell membranes in a nondestructive manner. HIV-1 TAT (see Ensoli et al., 67 J. Virol 277-287 (1993); Fawell et al., 91 Proc. Natl. Acad. Sci. USA 664-668 (1994); Schwarze et al., 285 Science 1569-1572 (1999)) and a small number of other non-viral proteins (see Jackson et al, 89 Proc. Natl. Acad. Sci. USA 10691-10695 (1992)) have also been attributed with intercellular trafficking properties

Sub-sequences of herpesviral VP22 protein with transport activity, and methods of testing these, have been described elsewhere. For example, see PCT International patent applications WO 97/05265, WO 98/04708, and WO 98/32866, each of which is incorporated herein by reference. Sub-sequences of herpesviral VP22 protein with transport activity include polypeptides corresponding to amino acids 60-301 and 159-301 of the full HSV1 VP22 sequence (1-301). A polypeptide consisting of amino acid residues 175-301 of the VP22 sequence has markedly less transport activity.

Deletion of the 34-amino acid C-terminal sequence from VP22 of HSV1 has been reported to abolish transport-activity (see PCT International patent applications WO 97/05265, WO 98/04708, and WO 98/32866). Thus this sequence region contains essential elements for transport activity.

Additionally, any other transport or carrier peptide(s) known to those skilled in the art may be used to effect intracellular deliver of the exogenous glucoincretin source. See Canadian Patent No. 5,067,481, which is incorporated herein by reference.

Moreover, one skilled in the art will recognize that any other method suitable for delivering an exogenous glucoincretin into the β-cells may also be employed. In one embodiment, the β-cells are isolated cells. An “isolated” β-cell is one that is separated from other cells, which are present in the natural source of the β-cells. Moreover, an “isolated” β-cell, can be substantially free of other cellular material, cultural medium, and/or contaminants.

Methods of Treating a Disorder Characterized by Impaired β-cell Function

The invention also involves a method of treating a disorder characterized by impaired β-cell function. This is accomplished by supplying a population of β-cells and a source of a glucoincretin to a patient suffering from the disorder. As discussed above, the glucoincretin may be a known cAMP-raising agent or a glucoincretin identified by any method known in the art, including, but not limited to, the methods described in Example 1, infra.

By a “disorder characterized by impaired β-cell function” is meant any disorder in which the pancreatic β-cells do not respond normally to an elevation of blood glucose levels. For example, such disorders includes, but are not limited to, non-insulin-dependent diabetes (Type II diabetes), impaired glucose tolerance (IGT), and insulin-dependent diabetes (Type I diabetes).

The patient to be treated is a mammal. Typically, the mammal is a human, but the mammal may also be a non-human primate, mouse, rat, dog, cat, horse, or cow.

The glucoincretin source and the β-cells may be encapsulated within a bioartificial organ. Administration of the β-cells and the glucoincretin source according to this invention may be achieved using any suitable delivery means, including:

(a) pump (see, e.g., Annals of Pharmacotherapy 27:912 (1993); Cancer 41:1270 (1993); Cancer Research 44:1698 (1984); incorporated herein by reference);

(b) microencapsulation (see e.g., U.S. Pat. Nos. 4,352,883; 4,353,888; and 5,084,350, herein incorporated by reference);

(c) continuous release polymer implants (see, e.g., U.S. Pat. No. 4,883,666, incorporated by reference);

(d) macroencapsulation (see, e.g., U.S. Pat. Nos. 5,284,761; 5,158,881, 4976,859, 5,800,828, 5,837,234; 5,798,113; 6,083,523; 5,955,095; and WO 95/05452, each incorporated by reference);

(e) naked or unencapsulated cell grafts (see, e.g., U.S. Pat. Nos. 5,082,670 and 5,618,531, each incorporated herein by reference);

(f) injection, either subcutaneously, intravenously, intraarterially, intramuscularly, or to another suitable site; and

(g) oral administration in capsule, liquid, tablet, pill, or prolonged release formulation.

The β-cells and the source of the glucoincretin can be incorporated into pharmaceutical compositions suitable for administration. Such compositions typically comprise β-cells and the gluocincretin and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the β-cell and the glucoincretin in the required amounts in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The dosage regimen is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Administration may be in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 15%, w/w or w/v.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The endogenous glucoincretin genes of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

As discussed in detail above, the source of the glucoincretin may be either an endogenous glucoincretin gene or an exogenous glucoincretin. If an endogenous glucoincretin is employed, the β-cell may be transfected with the endogenous gene using any of the methods outlined above. All or a portion of the β-cells may be transfected with an endogenous glucoincretin gene. For example, one, some, or all of the β-cells may be so transfected. Likewise, if an exogenous source is used, a transport or carrier peptide may be employed to introduce the glucoincretin into the β-cell.

A Population of Purified β-cells

The invention also includes a population of purified β-cells comprising an endogenous glucoincretin gene. As noted, the endogenous glucoincretin gene can be inserted into the cell in any manner known to those skilled in the relevant art. Suitable examples include: the use of polycations such as DEAE-dextran, calcium phosphate coprecipitation, electroporation, lipofection, retrovirus vectors, and microinjection.

The endogenous glucoincretin gene may encode a cAMP-raising agent. Such agents include, but are not limited to IBMX, GLP-1, GIP, glucagon, a cAMP-raising drug, a cAMP-raising enzyme, and a cAMP-raising hormone. Genes encoding other suitable cAMP-raising agents (or other glucoincretins) may also be employed. Determination of suitable glucoincretins (i.e, insulinotropic agents) may be accomplished according to the methods described in Example 1, infra. Moreover, other methods known to those skilled in the art may also be used to identify new agents.

Kits For Use in Treatment

The invention also concerns a kit comprising a population of purified β-cells and a source of a glucoincretin for use in the treatment of a disorder characterized by impaired β-cell function.

As above, a “disorder characterized by impaired β-cell function” is any disorder in which a subject's pancreatic β-cells do not respond normally to an elevation of blood glucose levels. For example, such disorders includes, but are not limited to, non-insulin-dependent diabetes (Type II diabetes), impaired glucose tolerance (IGT), and insulin-dependent diabetes (Type I diabetes).

In one embodiment, the invention provides for a kit comprising one or more reagents containing the purified β-cells and the source of the glucoincretin.

The glucoincretin may be a cAMP-raising agent. Such agents include, but are not limited to IBMX, GLP-1, GIP, glucagon, a cAMP-raising drug, a cAMP-raising enzyme, and a cAMP-raising hormone. Other suitable cAMP-raising agents (or other glucoincretins) may also be supplied. Determination of suitable glucoincretins (i.e, insulinotropic agents) may be made by any method known in the art.

The source of the glucoincretin may be either endogenous or exogenous to the β-cells. When the source of the glucoincretin is endogenous, the β-cells may be transfected with an endogenous glucoincretin gene. As described in detail above, any method of transfection known to those skilled in the art can be used.

Moreover, when an exogenous glucoincretin is used, the glucoincretin is delivered into the β-cells by means of a transport peptide. Methods of delivering the exogenous glucoincretin into the β-cell include the use of transport peptides, as described above.

The invention will be further described in the following examples, which do not limit the scope of the invention described in the claims.

### EXAMPLE 1

### Assays of Insulinotropic Activity

The insulinotropic property of a compound may be determined by providing that compound to animal cells, or injecting that compound into animals and monitoring the release of immunoreactive insulin (IRI) into the media or circulatory system of the animal, respectively. The presence of IRI is detected through the use of a radioimmunoassay, which can specifically detect insulin. Although any radioimmunoassay capable of detecting the presence of IRI may be employed, it is preferable to use a modification of the assay method of Albano, J. D. M., et al., Acta Endocrinol. 70:487-509 (1972). In this modification, a phosphate/albumin buffer with a pH of 7.4 was employed. The incubation was prepared with the consecutive condition of 500 μl of phosphate buffer, 50 μl of perfusate sample or rat insulin standard in perfusate, 100 μl of anti-insulin antiserum (Wellcome Laboratories; 1:40,000 dilution), and 100 μl of [¹²⁵I] insulin, giving a total volume of 750 μl in a 10×75-mm disposable glass tube. After incubation for 2-3 days at 4° C., free insulin was separated from antibody-bound insulin by charcoal separation. The assay sensitivity was 1-2 μU/ml. In order to measure the release of IRI into the cell culture medium of cells grown in tissue culture, one preferably incorporates radioactive label into proinsulin. Although any radioactive label capable of labeling a polypeptide can be used, it is preferable to use ³H leucine in order to obtain labeling of proinsulin. Labeling can be done for any period of time sufficient to permit the formation of a detectably labeled pool of proinsulin molecules; however, it is preferable to incubate cells in the presence of radioactive label for a 60-minute time period. Although any cell line capable of expressing insulin can be used for determining whether a compound has an insulinotropic effect, it is preferable to use rat insulinoma cells, and especially RIN-38 rat insulinoma cells. Such cells can be grown in any suitable medium; however, it is preferable to use DME medium containing 0.1% BSA and 25 mM glucose.

The insulinotropic property of a compound may also be determined by pancreatic infusion. The in situ isolated perfused rat pancreas preparation was a modification of the method of Penhos, J. C., et al. Diabetes 18:733-738 (1969). In accordance with such a method, fasted rats (preferably male Charles River strain albino rats), weighing 350-600 g, are anesthetized with an intraperitoneal injection of Amytal Sodium (Eli Lilly and Co., 160 ng/kg). Renal, adrenal, gastric, and lower colonic blood vessels are ligated. The entire intestine is resected except for about four cm of duodenum and the descending colon and rectum. Therefore, only a small part of the intestine is perfused, thus minimizing possible interference by enteric substances with glucagon-like immunoreactivity. The perfusate is preferably a modified Krebs-Ringer bicarbonate buffer with 4% dextran T70 and 0.2% bovine serum albumin (fraction V), and is preferably bubbled with 95% O₂ and 5% CO₂. A nonpulsatile flow, four-channel roller-bearing pump (Buchler polystatic, Buchler Instruments Division, Nuclear-Chicago Corp.) is preferably used, and a switch from one perfusate source to another is preferably accomplished by switching a three-way stopcock. The manner in which perfusion is performed, modified, and analyzed preferably follows the methods of Weir, G. C., et al., J. Clin. Investigat. 54:1403-1412 (1974), which is hereby incorporated by reference.

### EXAMPLE 2

### In vivo evaluation of the effect of GLP-1 injected intraperitoneally

In order to evaluate the in vivo effect of GLP-1 on the functionality of encapsulated and implanted β-cells, eight diabetic mice (6 C3H mice treated with streptozocin and 2 C57 mice treated with streptozocin were implanted intraperitoneally (IP) for 14 days with between 2×10⁶ and 8×10⁶ encapsulated CDM3D cells, which are types of β-TCtet cells. None of these mice demonstrated a correction of their hyperglycemia during the 14-day period prior to implantation.

The control group included three diabetic, non-implanted mice (2 C3H mice treated with streptozocin and 1 C57 mouse treated with streptozocin that had also exhibited hyperglycemia during the previous 14-day period. At time t=0, all mice received 0.1 nmol of GLP-1-gly8 IP (rather than wild-type GLP-1).

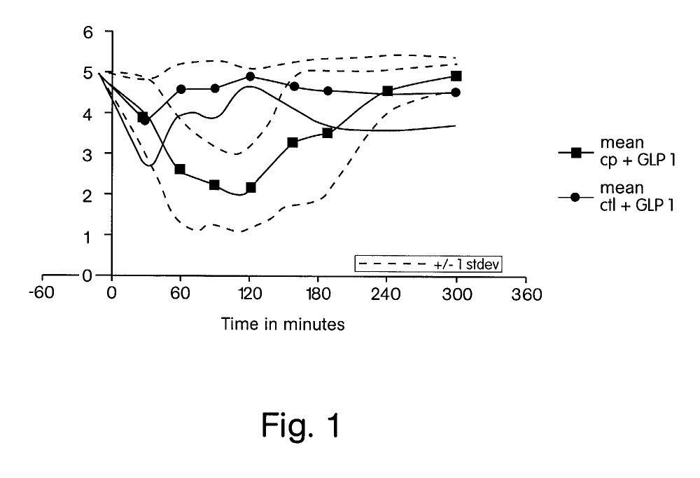

The results of this evaluation are shown in FIG. 1. According to FIG. 1 there is a large decrease in glycemia during the first two hours after GLP-1-gly8 injection for the test group (shaded boxes). A smaller (and shorter decrease) in glycemia was observed in the control group (shaded circles). The effect observed in the control group can probably be attributed to the few remaining islets (β-cells) in the pancreas of the mice that remain functional following streptozocin treatment. Moreover, the loss of the effect in the test group after 120 minutes may be attributed to the half-life of GLP-1-gly8, which is longer than the half-life of wild type GLP-1. Because the diffusion time within a macrocapsule is small, the relatively short half-life of the wild-type GLP-1 may play a role in the demise of encapsulated β-cells that are not supplied with a glucoincretin source.

### EXAMPLE 3

### Bioefficacy of GLP-1 Secreted by β-1081 Cells

In order to test the bioefficacy of GLP-1, which was believed to be secreted by β-1081 cells, the supernatant of the 1081 cells were kept for 4 hours in 25 mM KRBH. CDM5 cells (a type of β-cTCtet cell) were then stimulated with this supernatant. Next, the effect of this stimulation in cell membrane potential was measured using the bis-oxonol assay.

Those skilled in the art will recognize that GLP-1 has the effect of depolarizing the cell membrane upon application. This depolarization is the result of the stimulatory effect of GLP-1 on cAMP, which, as discussed above, leads to increased insulin secretion. The increased insulin secretion is the result of both the enhanced closing of ATP sensitive potassium (K⁺ ATP) channels in the membrane of the β-cell as well as the protein kinase A activation of distal exocytotic events.

### EXAMPLE 4

### Effect of the Supernatant of GLP-1 Secreting β-cells on the Membrane Potential of β-tcTet Cells

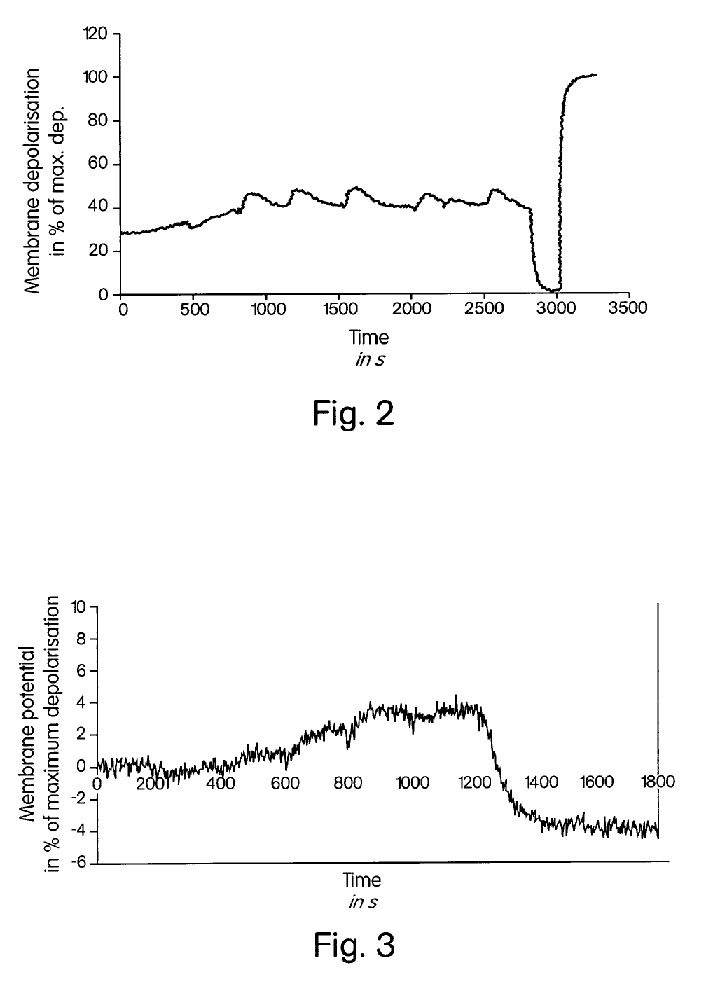

As shown in FIG. 2, a basal level of glucose (0.2 mM of glucose) was applied to 2×10⁶ CDM5 cells. At time t=100 seconds, the glucose concentration was increased to 2 mM, which resulted in membrane depolarization in response to glucose. At time t=400 s, the glucose concentration was raised to 16 mM, and, again, membrane depolarization in response to glucose was observed.

Next, 24 μl of the 1081 cell supernatant was added to the cells at times t=800 seconds, 1100 seconds, and 1500 seconds. At each time point, the addition of the supernatant resulted in a transient membrane depolarization.

At times t=2000 seconds and 2200 seconds, 1 nM and 2 nM of glucose, respectively, were added. Following this (at t=2500), an additional 24 μl of the supernatant was added. Once again, this resulted in a transient membrane depolarization. At t=2800 seconds, 400 μm Diazoxide was added, which produced maximum depolarization. Finally, at time t=3000 seconds, 60 mM KCl was added, which produced minimum depolarization of the membrane.

However, additional experimentation was necessary in order to determine whether the observed depolarization was the result of GLP-1 or some other compounds or agent found in the 1081 cell supernatant.

### EXAMPLE 5

### Effect of GLP1-gly8 and Exendine-(9-39) on Membrane Potential

As shown in FIG. 3, an experiment was performed to determine the effect of GLP-1-gly8 and Exendine-(9-39) on membrane potential. The cells were allowed to equilibrate in 8 mM glucose and 400 μM Diazoxide for 600 seconds. At 200, 400, 600, 800, and 1000 seconds, 1, 2, 4, 6, and 8 nM GLP-1-gly8, respectively, were added to the medium. For all GLP-1-gly8 concentrations except for 8 nM, the observed effect was the depolarization of the cell membrane. At 8 nM of GLP-1-gly8, a saturation of the effect was observed.

Following this, 20 nM and 40 nM Exendine-(9-39) (an inhibitor of the GLP-1 receptor) were added at 1200 seconds and 1600 seconds, respectively. Finally, at 1800 seconds, 60 mM KCl was added, which produced maximum depolarization of the cell membrane. Thus, following the addition of the inhibitor, the depolarizing response observed upon treatment with GLP-1-gly8 was stopped.

### EXAMPLE 6

### Effect of Exendine-(9-39) and GLP-1-gly8 on Membrane Potential

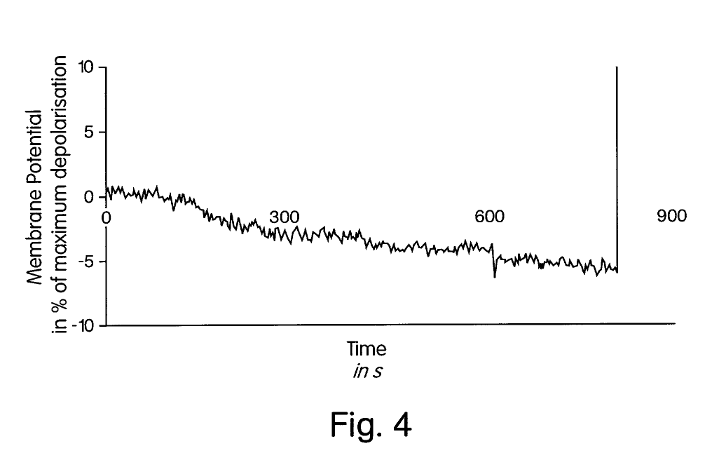

FIG. 4 shows the results of experiments performed to demonstrate the effects of Exendine-(9-39) and GLP-1-gly-8 on membrane potential. In this experiment, the cells were equilibrated at 8 mM glucose and 400 μM of diazoxide for 600 seconds. At t=100 seconds, 100 nM of Exendine-(9-39) was added. The addition of this inhibitor of GLP-1 receptor resulted in hyperpolarization of the cell membrane. This demonstrates the reverse agonist effect of Exendine-(9-39) since the compound is able to stop the basal effects of glucose. At t=600 seconds, 20 μl of water was added as a dilution control. Then, at t=600 seconds, 60 mM KCl was added, which produced maximum depolarization of the cell membrane.

As shown in FIG. 4, no response was observed with this commercial GLP-1-gly8 after exposure of the cells to Exendine-(9-39).

### EXAMPLE 7

### Effect of GLP1-gly8 and Exendine-(9-39) on Membrane Potential

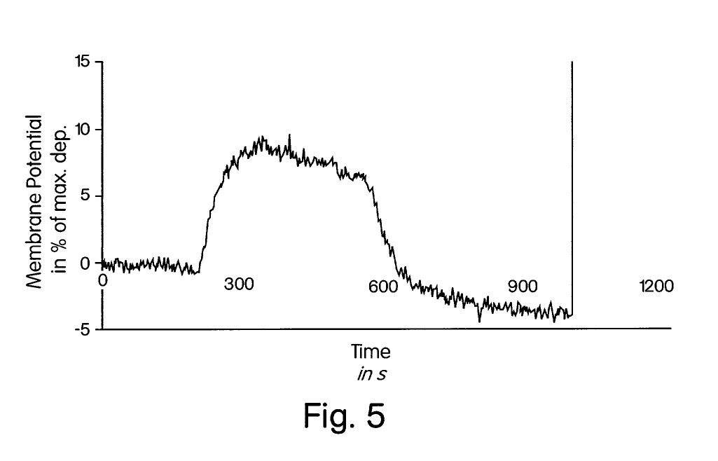

FIG. 5 shows the results of experiments performed to establish the effect of GLP-1 -gly8 and Exendine-(9-39) on membrane potential. Again, the cells were allowed to equilibrate at 8 mM glucose and 400 μM Diazoxide for 600 seconds. At 200 seconds, 24 μl of the supernatant of the GLP-1 secreting 1081 cells were added to the cells. This resulted in a transient depolarization of the cell membrane. After 600 seconds, 100 nM Exendine-(9-39) was added, resulting first in repolarization and then in hyperpolarization of the cell membrane. At 1500 seconds, 24 μl of the supernatant was added. However, this did not result in any depolarization of the cell membrane, since its actions were blocked by the GLP-1 receptor inhibitor. Finally, at 1000 seconds, 60 mM KCl was added, which produced maximum depolarization of the cell membrane.

### EXAMPLE 8

### Effect of Exendine and the Supernatant of GLP-1 Secreting Cells on Membrane Potential

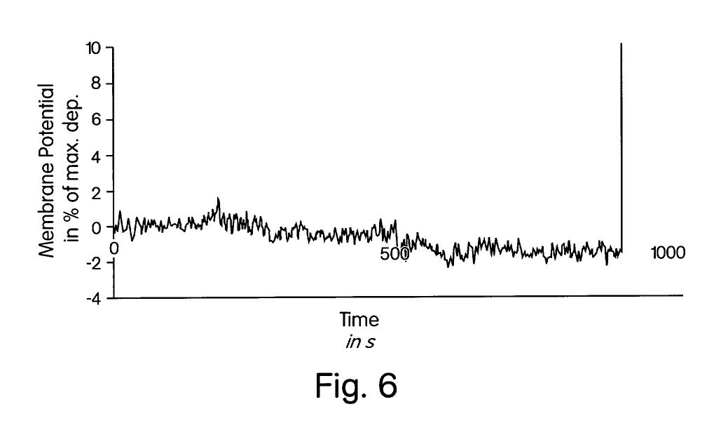

Exendine (9-39) is an inhibitor of GLP-1 activity. As shown in FIG. 3, 2×10⁶ cells were placed in 8 mM glucose and 400 μM Diazoxide, which produced the maximum hyperpolarization. In this experiment, the equilibration period lasted from time t=600 seconds to time t=0. At t=200 seconds, 100 mM of Exendine (9-39) was added to the cells. This resulted in hyperpolarization of the cell membrane. The addition of 24 μl of supernatant at t=500 seconds and 700 seconds did not produce depolarization of the membrane, presumably because the action of the GLP-1 present in the supernatant was blocked by the addition of Exendine (9-39). Finally, the addition of 60 mM KCl at time t=900 seconds produced the maximum depolarization. These results are shown in FIG. 6.

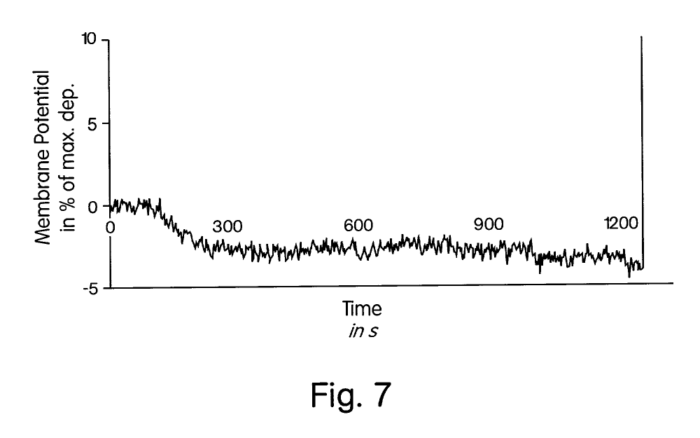

The results of a similar experiment are shown in FIG. 7. There, the cells equilibrated at 8 mM glucose and 400 μM Diazoxide for 600 seconds. This was followed by 20 nM of Exedine-(9-39) at 100 seconds, which resulted in hyperpolarization of the cell membrane. Then, at 600, 700, and 1000 seconds, 24 μl of the supernatant of the 1081 cells was added. None of these additions resulted in depolarization of the cell membrane. Finally, 60 mM KCl was added at 1000 seconds. This produced the maximum depolarization of the membrane.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of enhancing the responsiveness of a β-cell to glucose comprising providing a source of a glucoincretin that increases the responsiveness of the cell to glucose.

2. The method of claim 1 wherein the source of the glucoincretin is an endogenous glucoincretin gene.

3. The method of claim 2 wherein the β-cell is transfected with the endogenous glucoincretin gene.

4. The method of claim 1 wherein the source of the glucoincretin is exogenous to the β-cell.

5. The method of claim 4 wherein the glucoincretin is delivered into the β-cell by a transport peptide.

6. The method of claim 1 wherein the glucoincretin is a cAMP-raising agent.

7. The method of claim 6 wherein the cAMP-raising agent is selected from the group consisting of IBMX, GLP-1, GIP, glucagon, a cAMP-raising drug, a cAMP-raising enzyme, and a cAMP-raising hormone.

8. The method of claim 1 wherein the β-cell is isolated.

9. A method of treating a disorder characterized by impaired β-cell function comprising supplying a population of β-cells and a source of a glucoincretin to a patient suffering from said disorder.

10. The method of claim 9 wherein the population of β-cells and the source of the glucoincretin are encapsulated within a bioartificial organ.

11. The method of claim 10 wherein the bioartificial organ comprises a semipermeable jacket and a core containing the population of β-cells and the source of the glucoincretin.

12. The method of claim 9 wherein the source of the glucoincretin is an endogenous glucoincretin gene.

13. The method of claim 12 wherein the population of β-cells are transfected with the endogenous glucoincretin gene.

14. The method of claim 12 wherein a portion of the population of β-cells are transfected with the endogenous glucoincretin gene.

15. The method of claim 9 wherein the source of the glucoincretin is exogenous to the β-cell.

16. The method of claim 15 wherein the glucoincretin is delivered into the β-cell by a transport peptide.

17. The method of claim 9 wherein the glucoincretin is a cAMP-raising agent.

18. The method of claim 17 wherein the cAMP-raising agent is selected from the group consisting of IBMX, GLP-1, GIP, glucagon, a cAMP-raising drug, a cAMP-raising enzyme, and a cAMP-raising hormone.

19. A population of purified β-cells comprising an endogenous glucoincretin gene.

20. The population of purified β-cells of claim 19 wherein the glucoincretin is a cAMP-raising agent.

21. The population of purified β-cells of claim 20 wherein the cAMP-raising agent is selected from the group consisting of IBMX, GLP-1, GIP, glucagon, a cAMP-raising drug, a cAMP-raising enzyme, and a cAMP-raising hormone.

22. A kit comprising a population of purified β-cells and a source of a glucoincretin for use in the treatment of a disorder characterized by impaired β-cell function.

23. The kit according to claim 22 wherein the source of the glucoincretin is an endogenous glucoincretin gene.

24. The method of claim 23 wherein the β-cell is transfected with the endogenous glucoincretin gene.

25. The method of claim 22 wherein the source of the glucoincretin is exogenous to the β-cell.

26. The method of claim 25 wherein the glucoincretin is delivered into the β-cell by a transport peptide.

27. The method of claim 22 wherein the glucoincretin is a cAMP-raising agent.

28. The method of claim 27 wherein the cAMP-raising agent is selected from the group consisting of IBMX, GLP-1, GIP, glucagon, a cAMP-raising drug, a cAMP-raising enzyme, and a cAMP-raising hormone.
